(19) United States
(12) Patent Application Publication
(10) Pub. No.: US 2012/0088829 A1
(43) Pub. Date: Apr. 12, 2012

(54) Formulations of Ubiquinol and Resveratrol Esters

(75) Inventor: Volker Berl, New York, NY (US)

(73) Assignee: MyCell Holdings Limited

(21) Appl. No.: 13/248,853

(22) Filed: Sep. 29, 2011

Related U.S. Application Data
(60) Provisional application No. 61/387,788, filed on Sep. 29, 2010.

(51) Int. Cl. A61K 31/216; A61P 39/06

(52) U.S. Cl. 514/548

(57) ABSTRACT

Disclosed herein are stabilized aqueous formulations comprising a ubiquinol ester/diester or resveratrol ester, and a micelle-forming surfactant, and methods for preparing the formulations. In one embodiment, the formulation remains substantially clear and stable when stored at or below room temperature for a period of at least 12 months.

This application claims the benefit of U.S. Provisional Application No. 61/387,788 filed Sep. 29, 2010, which is incorporated herein by reference.

### SUMMARY OF THE PRESENT APPLICATION

A need exists for novel methods of preparing formulations comprising food, beverage, pharmaceutical, nutraceutical, cosmetics, or cosmeceutical products containing nutritional products. The following embodiments, aspects and variations thereof are exemplary and illustrative are not intended to be limiting in scope.

In one embodiment, there is provided a formulation or a composition comprising:

a) a ubiquinol ester/diester or a resveratrol ester, or mixtures thereof; and

b) a solubilizing agent comprising the Formula (I):

Y¹—[L¹]ₐ—Z (I)

wherein:
a is 0 and 1;
L¹ is a linker moiety that covalently links the hydrophobic moiety Z and the hydrophilic moiety Y¹;
Y¹ is a linear or branched hydrophilic moiety comprising at least one polymeric moiety independently selected from poly(alkylene oxides) and polyalcohols; and
Z is a hydrophobic moiety.

In one aspect of the embodiment, the ubiquinol ester/diester or the resveratrol ester is selected from the group consisting of amino acid esters, nutritional acid esters, C₂₋₂₀ alkyl ester and C₆₋₂₀ aryl ester, and mixtures thereof. In another aspect, the ubiquinol ester/diester or the resveratrol ester is selected from the group consisting of the C₂₋₁₂ alkyl ester, the C₂₋₁₀ alkyl ester, the C₂₋₅ alkyl ester and the C₂-C₃ alkyl ester, and mixtures thereof. In another aspect, the nutritional acid esters are selected from the group consisting of omega-3, omega-6, and omega-9 fatty acids, α-linolenic acid (ALA), stearidonic acid, eicosatetraenoic acid, eicosapentaenoic acid (EPA), docosapentaenoic acid, docosahexaenoic acid (DHA), linoleic acid, gamma-linolenic acid, eicosadienoic acid, dihomo-gamma-linolenic acid, arachidonic acid, docosadienoic acid, adrenic acid, docosapentaenoic acid, oleic acid, eicosenoic acid, mead acid, erucic acid, nervonic acid, vitamin B, vitamin B-3, biotin, folic acid, pantothenic acid, para-amino benzoic acid and taurine, and mixtures thereof. In another aspect, the composition further comprises water to form an aqueous composition, wherein the aqueous composition is a substantially clear, water soluble composition. In yet another aspect, the solubilizing agent comprises the Formula (I), wherein:
Z is selected from the group consisting of sterols, tocopherols, tocotrienol and ubiquinol ester/diester and derivatives or homologues thereof;
L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene; and
Y¹ is a linear or branched hydrophilic moiety including at least one polymeric moiety, wherein each polymeric moiety is a member independently selected from poly(alkylene oxides) and polyalcohols.

In one variation of the above composition:
Y¹ is selected from the group consisting of poly(alkylene oxides) and monoethers therefrom, polyalcohols, polysaccharides, polyamino acids, polyphosphoric acids, polyamines and derivatives thereof; and
L¹ is selected from the group consisting of a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group selected from the group consisting of ether, thioether, ester, carboxamide, sulfonamide, carbonate and urea groups.

In another aspect of the composition, the solubilizing agent is TPGS (polyoxyethanyl-a-tocopheryl succinate) or TPGS-1000 (polyoxyethanyl-a-tocopheryl succinate-1000). In one variation of each of the above, the composition may further comprise a stabilizing agent.

In another embodiment, there is provided a method for stabilizing a bioactive compound selected from the group consisting of ubiquinol ester/diester and resveratrol ester, and mixtures thereof, in an aqueous solution comprising contacting the bioactive compound, with a composition comprising a micelle-forming surfactant for a sufficient period of time to dissolve the bioactive compound. In one aspect of the method, the bioactive compound is selected from the group consisting of a ubiquinol ester/diester and resveratrol ester selected from the group consisting of the C₂₋₂₀ alkyl ester, the C₂₋₁₀ alkyl ester, the C₂₋₆ alkyl ester or the C₂₋₃ alkyl ester, and mixtures thereof. In another aspect of the method, the micelle-forming surfactant is TPGS (polyoxyethanyl-a-tocopheryl succinate) or TPGS-1000 (polyoxyethanyl-a-tocopheryl succinate-1000). In one variation of the above methods, the composition further comprises a stabilizing agent. In another aspect, the method provides contacting the bioactive compound with the composition comprising a micelle-forming surfactant for a sufficient period of time to dissolve the bioactive compound is performed at an elevated temperature.

In another embodiment, there is provided a method for increasing the bioavailability and/or the absorption of ubiquinol or resveratrol, or mixtures thereof, in a mammal, comprising: preparing an absortion enhanced bioactive formulation of a ubiquinol ester/diester or a resveratrol ester, or mixtures thereof, wherein the bioactive formulation comprises:

a) a ubiquinol ester/diester or a resveratrol ester or mixtures thereof; and

b) a solubilizing agent comprising the Formula (I):

Y¹—[L¹]ₐ—Z (I)

wherein:

a is 0 and 1;
L¹ is a linker moiety that covalently links the hydrophobic moiety Z and the hydrophilic moiety Y¹;
Y¹ is a linear or branched hydrophilic moiety comprising at least one polymeric moiety independently selected from poly(alkylene oxides) and polyalcohols; and
Z is a hydrophobic moiety; and

administering an effective amount of the formulation in the mammal to enhance the bioavailability or absorption of the ubiquinol or resveratrol in the mammal by a factor of at least two when compared to the administration of the same quantity of a ubiquinol or a resveratrol in the absence of the absorption enhanced bioactive formulation. In one aspect of the above method, the enhancement of bioavailability or absorption of the ubiquinol or resveratrol in the mammal is by a factor of at least two to ten. In another aspect of the method, the ubiquinol ester/diester or resveratrol ester is selected from the group consisting of the C₂₋₂₀ alkyl ester, the C₂₋₁₀ alkyl ester, the C₂₋₆ alkyl ester or the C₂₋₃ alkyl ester, and mixtures thereof. In another aspect of the above method, the solubilizing agent is TPGS (polyoxyethanyl-a-tocopheryl succinate) or TPGS-1000 (polyoxyethanyl-a-tocopheryl succinate-1000).

In another embodiment, the water-soluble formulation further comprises a water soluble antioxidant. In another embodiment, the water-soluble formulation further comprises a lipophilic antioxidant.

In another aspect of the formulation, the solubilizing agent comprises the Formula (I), wherein:
Z is selected from the group consisting of sterols, tocopherols and tocotrienol and esters and derivatives or homologues thereof;
L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene; and
Y¹ is a linear or branched hydrophilic moiety including at least one polymeric moiety, wherein each polymeric moiety is a member independently selected from poly(alkylene oxides), polyalcohols, and polyalcohol monoethers.

In another aspect of the water-soluble formulation: Y¹ is selected from the group consisting of poly(alkylene oxides) and monoethers derived therefrom, polyalcohols, polysaccharides, polyamino acids, polyphosphoric acids, polyamines and derivatives thereof; and L¹ is selected from the group consisting of a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group selected from the group consisting of ether, thioether, ester, carboxamide, sulfonamide, carbonate and urea groups. In one variation, the solubilizing agent is TPGS (polyoxyethanyl-a-tocopheryl succinate). In another variation, the TPGS is the natural vitamin-E TPGS.

In another embodiment, there is provided a method for preparing a composition comprising a ubiquinol ester/diester or resveratrol ester, or mixtures thereof, in an aqueous solution comprising contacting a ubiquinol ester/diester or resveratrol ester, or mixtures thereof, with a composition comprising a solubilizing agent (or micelle-forming surfactant), at an elevated temperature, and for a sufficient period of time to dissolve the lipophilic bioactive compound. In another aspect, the solubilizing agent is TPGS (polyoxyethanyl-a-tocopheryl succinate). In a particular variation, the solubilizing agent is TPGS-1000.

In one aspect of the formulation, the ubiquinol ester/diester is selected from the group consisting of C₂₋₂₀ alkyl ester, C₂₋₁₀ alkyl ester, C₂₋₆ alkyl ester and C₂₋₃ alkyl ester, and mixtures thereof. In one embodiment, the ester is a C₂ alkyl ester. Accordingly, in another embodiment, there is provided a food, beverage, pharmaceutical or nutraceutical product comprising the aqueous formulation of the above.

In another aspect of the formulation, the resveratrol ester is selected from the group consisting of mono-ester, diester or triester that is the C₂₋₂₀ alkyl ester, C₂₋₁₀ alkyl ester, C₂₋₆ alkyl ester and C₂₋₃ alkyl ester, and mixtures thereof. In one embodiment, the ester is a C₂ alkyl ester. Accordingly, in another embodiment, there is provided a food, beverage, pharmaceutical or nutraceutical product comprising the aqueous formulation of the above.

In addition to the exemplary embodiments, aspects and variations described above, further embodiments, aspects and variations will become apparent by reference to the drawings and figures and by examination of the following descriptions.

### DETAILED DESCRIPTION OF THE PRESENT APPLICATION DEFINITIONS

Unless specifically noted otherwise herein, the definitions of the terms used are standard definitions used in the art of organic synthesis and pharmaceutical sciences. Exemplary embodiments, aspects and variations are illustratived in the figures and drawings, and it is intended that the embodiments, aspects and variations, and the figures and drawings disclosed herein are to be considered illustrative and not limiting.

### DEFINITIONS

The term “vitamin C derivative” as used herein means any compound that releases ascorbic acid (vitamin C) in vivo or in vitro, as well as solvates, hydrates and salts thereof. The term also includes vitamin C analogs wherein one or more of the hydroxyl groups of vitamin C are substituted with another moiety and wherein the vitamin C analog essentially retains the stabilizing activity of vitamin C in vitro or in vivo.

The term “monoterpene” as used herein, refers to a compound having a 10-carbon skeleton with non-linear branches. A monoterpene refers to a compound with two isoprene units connected in a head-to-tail manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “solubilizing agent” is used interchangeably with the term “surfactant”. In one embodiment, the solubilizing agent is a nonionic, amphiphilic molecule, wherein the term amphiphilic means that the molecule includes at least one hydrophobic (e.g., lipid-soluble) moiety, such as a moiety derived from a tocopherol, a sterol, a simple sugar or carbohydrate, or a quinone (or derived hydroquinone, such as in the case of ubiquinone and ubiquinol) and at least one hydrophilic (e.g., water-soluble) moiety, such as polyethylene glycol.

As used herein, the terms “stabilizer”, and “antioxidant”, are recognized in the art and refer to synthetic or natural substances that prevent or delay the oxidative or free radical or photo induced deterioration of a compound, and combinations thereof. Exemplary stabilizers include tocopherols, flavonoids, catechins, superoxide dismutase, lecithin, gamma oryzanol; vitamins, such as vitamins A, C (ascorbic acid) and E (tocopherol and tocopherol homologues and isomers, especially alpha and gamma-tocopherol) and beta-carotene (or related carrotenoids); natural components such as camosol, carnosic acid and rosmanol found in rosemary and hawthorn extract, proanthocyanidins such as those found in grape seed or pine bark extract, and green tea extract. In one variation, the vitamin E includes all 8-isomers, and also include d,l-tocopherol or d,l-tocopherol acetate (i.e., dl, dl, dl-tocopherol). In one variation, the vitamin E is the d,d,d-alpha form of vitamin E. In another variation, the vitamin E includes natural, synthetic and semi-synthetic compositions and combinations thereof. In one variation, the vitamin E is the naturally occurring vitamin E.

The term “water-soluble” when referring to a formulation or compositions of the present application, means that the formulation when added to an aqueous medium (e.g., water, original beverage) dissolves in the aqueous medium to produce a solution that is essentially clear. In one example, the formulation dissolves in the aqueous medium without heating the resulting mixture above ambient temperature (e.g., 25° C.). The term “essentially clear” is defined herein.

The term “aqueous formulation” refers to a formulation of the present application including at least about 5% (w/w) water. In one example, an aqueous formulation includes at least about 10%, at least about 20%, at least about 30% at least about 40% or at least about 50% (w/w) of water.

The term “bioactive” refers to compounds and compositions of the present application. For example, a bioactive molecule is any compound having in vivo and/or in vitro biological activity. In one embodiment, the bioactive or bioactive molecule is a fatty acids, such as ubiquinol ester. In another embodiment, the bioactive is resveratrol or resveratrol ester. Bioactive molecules or compositions also include those, which are suspected in the art to have biological activity (e.g., to have a positive effect on human health and/or nutrition). In one example, the biological activity is a desirable biological activity but can be accompanied by undesirable side-effects. Compounds with biological activity include pharmaceuticals, neutraceuticals and dietary supplements.

The terms “ubiquinol ester(s)” are used interchangeable with “ubiquinol diester(s).” In one aspect, unless specified otherwise as being a mixture, the term “ubiquinol ester” may be used interchangeably with the terms ubiquinol esters or ubiquinol diester to mean the same compound. In another aspect, the term “ubiquinol ester/diester” means ubiquinol monoester, ubiquinol diester or a mixture of ubiquinol monoester and diester. In certain embodiments of the present application, the non-naturally occurring ubiquinol ester used in the compositions of the present application comprise the C₂₋₂₀ alkyl ester, the C₂₋₁₀ alkyl ester, the C₂₋₆ alkyl ester, the C₂₋₃ alkyl ester or the C₂ alkyl ester, or mixtures thereof. Accordingly, the ubiquinol monoester may have the ester functionalized at the 1-carbon or at the 4-carbon (based on the ubiquinone nomenclature). In another embodiment, the ubiquinol diester may have the same ester (i.e., both esters being the same (e.g. a C₂ alkyl ester) at the 1-carbon and the 4-carbon), or the ubiquinol diester may have different esters (e.g., having a C₂ alkyl ester at the 1-carbon and a C₃ alkyl ester at the 4-carbon etc. . . . ). In the later case, such diesters are referred to as being a mixture of esters.

Accordingly, as used herein, unless otherwise noted, the term “ubiquinol ester” as used in each aspect, variations and embodiments of the formulations of the present application include their monoesters, diesters, and mixtures thereof, as defined herein.

The terms “resveratrol ester(s)” as used herein include resveratrol mono-ester, resveratrol diester(s) and resveratrol tri-ester(s). Resveratrol is also referred to as 3,5,4′-trihydroxy-trans-stilbene. In certain embodiments of the present application, the non-naturally occurring resveratrol ester used in the compositions of the present application comprise the C₂₋₂₀ alkyl ester, the C₂₋₁₀ alkyl ester, the C₂₋₆ alkyl ester, the C₂₋₃ alkyl ester or the C₂ alkyl ester. Accordingly, as used herein, unless otherwise noted, the term “resveratrol ester” as used in each aspect, variations and embodiments of the formulations of the present application include their mono-esters, diesters and tri-esters, and mixtures thereof, as defined herein. For example, as shown below, R^{x}, R^{y} and R^{z} may be C₁₋₂₀ alkyl, C₁₋₁₀ alkyl, C₂₋₅ alkyl, C₂₋₃ alkyl or C₂ alkyl that forms the corresponding C₂₋₂₁ alkyl ester, C₂₋₁₁ alkyl ester, C₃₋₆ alkyl ester, C₃₋₄ alkyl ester or the C₃ alkyl ester, respectively. These alkyl esters may be the mono-esters, diesters and tri-esters and mixtures thereof. Accordingly, a monoester may be at the 3-carbon, the 5-carbon or the 4′-carbon. A diester of resveratrol may include various regioisomers, such as the 3,5-diester, the 3,4′-diester etc. The diester may have the same esters or different esters at each of the 3-, 5- and 4′-carbon positions. As provided herein, the tri-esters may also include the same esters (e.g., the 3-, 5- and 4′-carbon positions are all C₂ alkyl esters), or different esters (e.g., the 3-carbon is a C₂ alkyl ester, the 5-carbon is a C₃ alkyl ester, the 4′-carbon is a C₂ alkyl ester (or a C₃ alkyl ester or a C₄ alkyl ester, etc . . . )). As used herein, such permutations may be considered to be mixtures of esters.

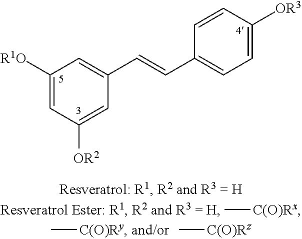

The terms “amino acid esters” refer to natural and unnatural amino acids that form the carboxylic esters with ubiquinol or resveratrol. The amino acids are well known in the art, and non-exclusive examples of amino acids include alanine, arginine, asparagine aspartic acid, cysteine, glutamic acid, glycine, histidine, isoleucine, leucine, lysine, methionine, phenylalanine, proline, serine, threonine, tryptophan, tyrosine, valine and taurine. These amino acid esters may also include the mono-esters, diesters and tri-esters and mixtures thereof.

The terms “nutritional acid esters” refer to nutritional compounds or supplements containing a carboxylic acid group that may form an ester with ubiquinol or resveratrol. Non-exclusive examples of nutritional acid esters include vitamin B, vitamin B-3, biotin, folic acid, fatty acids, fatty acid esters, pantothenic acid, para-amino benzoic acid and taurine.

The term “pharmaceutical”, “pharmaceutical composition” or pharmaceutical formulation” encompasses “neutraceutical” also referred to as “nutraceutical”), “neutraceutical composition” or “neutraceutical formulation”, respectively. Neutraceutical formulations or neutraceutical compositions may include a pharmaceutically acceptable carrier, such as those described herein.

The term “neutraceutical” or “nutraceutical” is a combination of the terms “nutritional” and “pharmaceutical”. It refers to a composition, which is known or suspected in the art to positively affect human nutrition and/or health.

The term “beverage” describes any water-based liquid, which is suitable for human consumption (i.e., food-grade). A typical beverage of the present application is any “original beverage” in combination with at least one bioactive lipophilic molecule of the present application. “Original beverage” can be any beverage (e.g., any marketed beverage). The term “original beverage” includes beers, carbonated and non-carbonated waters (e.g., table waters and mineral waters), flavored waters (e.g., fruit-flavored waters), mineralized waters, sports drinks (e.g., Gatorade®), smoothies, neutraceutical drinks, filtered or non-filtered fruit and vegetable juices (e.g., apple juice, orange juice, cranberry juice, pineapple juice, lemonades and combinations thereof) including those juices prepared from concentrates. Exemplary juices include fruit juices having 100% fruit juice (squeezed or made from concentrate), fruit drinks (e.g., 0-29% juice), nectars (e.g., 30-99% juice). The term “original beverage” also includes fruit flavored beverages, carbonated drinks, such as soft-drinks, fruit-flavored carbonates and mixers. Soft drinks include caffeinated soft drinks, such as coke (e.g., Pepsi Cola®, Coca Cola®) and any “diet” versions thereof (e.g., including non-sugar sweeteners). The term “original beverage” also includes teas (e.g., green and black teas, herbal teas) including instant teas, coffee, including instant coffee, chocolate-based drinks, malt-based drinks, milk, drinkable dairy products and beer. The term “original beverage” also includes any liquid or powdered concentrates used to make beverages.

The term “clear beverage” (e.g., clear juice) means any beverage clear (e.g., transparent) to the human eye. Typical clear beverages include carbonated or non-carbonated waters, energy drinks, soft drinks, such as Sprite®, Coke® or root beer, filtered juices and filtered beers. Typical non-clear beverages include orange juice with pulp and milk.

The term “non-alcoholic beverage” includes beverages containing essentially no alcohol. Exemplary non-alcoholic beverages include those listed above for the term “beverage”. The term “non-alcoholic beverage” includes beers, including those generally referred to as “non-alcoholic beers”. In one example, the non-alcoholic beverage includes less than about 10% alcohol by volume. In another example, the non-alcoholic beverage includes less than about 9% or less than about 8% alcohol by volume. In yet another example, the non-alcoholic beverage includes less than about 7%, less than about 6% or less than about 5% alcohol by volume.

The term “essentially stable to chemical degradation” refers to a bioactive molecule of the present application as contained in a formulation (e.g., aqueous formulation), beverage or other composition of the present application. In one example, “essentially stable to chemical degradation” means that the molecule is stable in its original (e.g., reduced) form and is not converted to another species (e.g., oxidized species; any other species including more or less atoms; any other species having an essentially different molecular structure), for example, through oxidation, cleavage, rearrangement, polymerization and the like, including those processes induced by light (e.g., radical mechanisms). Examples of chemical degradation include oxidation and/or cleavage of double bonds in unsaturated fatty acids and light-induced rearrangements of unsaturated molecules. The molecule is considered to be essentially stable when the concentration of its original (e.g., reduced) form in the composition (e.g., aqueous formulation) is not significantly diminished over time. For example, the molecule is essentially stable when the concentration of the original form of the molecule remains at least 80% when compared with the concentration of the original form of the molecule at about the time when the composition is prepared. In another example, the molecule is essentially stable when the concentration of the original form remains at least about 85%, at least about 90% or at least about 95% of the original concentration. For example, an aqueous composition containing ubiquinol ester or resveratrol ester at a concentration of about 50 mg/ml is considered essentially stable for at least 90 days when, at the end of the 90 days, the concentration of ubiquinol ester or resveratrol ester in the aqueous composition remains at least about 40 mg/ml (80% of 50 mg/ml).

The term “essentially clear” is used herein to describe the compositions (e.g., formulations) of the present application. For example, the term “essentially clear” is used to describe an aqueous formulation or a beverage of the present application. In one example, clarity is assessed by the normal human eye. In this example, “essentially clear” means that the composition is transparent and essentially free of visible particles and/or precipitation (e.g., not visibly cloudy, hazy or otherwise non-homogenous). In another example, clarity, haziness or cloudiness of a composition is assessed using light scattering technology, such as dynamic light scattering (DLS), which is useful to measure the sizes of particles, e.g., micelles, contained in a composition. In one example, “essentially clear” means that the median particle size as measured by DLS is less than about 100 nm. For example, when the median particle size is less than 100 nm the liquid appears clear to the human eye. In another example, “essentially clear” means that the median particle size is less than about 80 nm. In yet another example, “essentially clear” means that the median particle size is less than about 60 nm. In a further example, “essentially clear” means that the median particle size is less than about 40 nm. In another example, “essentially clear” means that the median particle size is between about 20 and about 30 nm. A person of skill in the art will know how to prepare a sample for DLS measurement. For example, in order to prepare a sample (e.g., formulation of the present application) for a DLS measurement, the sample is typically diluted so that the concentration of the solubilizing agent in the diluted sample is between about 1 mM (10⁻³ M) and 0.01 mM (10⁻⁵ M). In another example, the solubilizing agent (i.e., TPGS) is present in a concentration that is above the critical micelle concentration (CMC) (i.e., the concentration that allows for spontaneous formation of micelles in water). For example, a typical CMC for TPGS in water is about 0.1 to about 0.5 mg/ml. A person of skill in the art will be able to select suitable concentrations in order to successfully measure particle sizes in a formulation of the present application.

Alternatively, clarity, haziness or cloudiness of a composition of the present application can be determined by measuring the turbidity of the sample. This is especially useful when the composition is a beverage (e.g., water, soft-drink etc.). In one example, turbidity is measured in FTU (Formazin Turbidity Units) or FNU (Formazin Nephelometric Units). In one example, turbidity is measured using a nephelometer. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the present application (e.g., a beverage of the present application) is “essentially clear” when the turbidity is not more than about 500% higher than the control (original beverage without an added lipophilic bioactive molecule of the present application, but optionally including a solubilizing agent of the present application, i.e., TPGS). For example, the turbidity of a sample of flavored water is measured to be 2.0 ntu and the turbidity of another sample containing the same flavored water in combination with a fatty acids is measured to be at or below about 8.0 ntu (2.0 ntu+200%=8.0 ntu), then the fatty acids sample is considered to be essentially clear. In another example, a composition of the present application is “essentially clear” when the turbidity is not more than about 300% higher than the control. In yet another example, a composition of the present application is “essentially clear” when the turbidity is not more than about 200%, about 150% or about 100% higher than the control. In a further example, a composition of the present application is “essentially clear” when the turbidity is not more than about 80%, about 60%, about 40%, about 20% or about 10% higher than the control.

The term “emulsion” as used herein refers to a ubiquinol ester or resveratrol ester emulsified (solubilized) in an aqueous medium using a solubilizing agent of the present application. In one example, the emulsion includes micelles formed between ubiquinol ester or resveratrol ester and the solubilizing agent. When those micelles are sufficiently small, the emulsion is essentially clear. Typically, the emulsion will appear clear (e.g., transparent) to the normal human eye, when those micelles have a median particle size of less than 100 nm. In one example, the micelles in the emulsions of the present application have median particle sizes below 60 nm. In a typical example, micelles formed in an emulsion of the present application have a median particle size between about 20 and about 30 nm. In another example, the emulsion is stable, which means that separation between the aqueous phase and the lipophilic component does essentially not occur (e.g., the emulsion stays clear). A typical aqueous medium, which is used in the emulsions of the present application, is water, which may optionally contain other solubilized molecules, such as salts, coloring agents, flavoring agents and the like. In one example, the aqueous medium of the emulsion does not include an alcoholic solvent, such as ethanol or methanol.

The term “micelle” is used herein according to its art-recognized meaning and includes all forms of micelles, including, for example, spherical micelles, cylindrical micelles, worm-like micelles and sheet-like micelles, and vesicles, formed in water, or mostly water.

The term “flavonoid” as used herein is recognized in the art. The term “flavonoid” includes those plant pigments found in many foods that are thought to help protect the body from disease (e.g., cancer). These include, for example, epi-gallo catechin gallate (EGCG), epi-gallo catechin (EGC) and epi-catechin (EC).

The term “tocopherol” includes all tocopherols, including alpha-, beta-, gamma- and delta tocopherol. The term “tocopherol” also includes tocotrienols.

Where substituent groups are specified by their conventional chemical formulae, written from left to right, they equally encompass the chemically identical substituents, which would result from writing the structure from right to left, e.g., —CH₂O— is intended to also recite and include —OCH₂—.

The term “alkyl,” by itself or as part of another substituent, means, unless otherwise stated, a straight or branched chain, or cyclic hydrocarbon radical, or combination thereof, which can be fully saturated, mono- or polyunsaturated and can include di- and multivalent radicals, having the number of carbon atoms designated (i.e. C₁-C₆ means one to six carbons, C₂-C₆ means two to six carbons). Examples of saturated hydrocarbon radicals having C₂-C₁₀ that are the esters (i.e., —COC₁-C₉, wherein the carbon atom of the carbonyl group is considered to be one of the carbon atoms of the “C₂-C₁₀ ester” group) or diesters of ubiquinol include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” with the difference that the heteroalkyl group, in order to qualify as an alkyl group, is linked to the remainder of the molecule through a carbon atom. Alkyl groups that are limited to hydrocarbon groups are termed “homoalkyl”.

The term “alkenyl” by itself or as part of another substituent is used in its conventional sense, and refers to a radical derived from an alkene, as exemplified, but not limited, by substituted or unsubstituted vinyl and substituted or unsubstituted propenyl. Typically, an alkenyl group will have from 1 to 24 carbon atoms. In one aspect, the alkenyl groups have from 1 to 10 carbon atoms.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms. In one aspect, the groups may have 10 or fewer carbon atoms. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, S, B and P and wherein the nitrogen and sulfur atoms can optionally be oxidized and the nitrogen heteroatom can optionally be quaternized. The heteroatom(s) can be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′— and —OC(O)W—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent can be attached to the remainder of the molecule directly or through a linker. An exemplary linker is alkylene. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, 1-piperazinyl, 2-piperazinyl, and the like.

The terms “halo” or “halogen,” by themselves or as part of another substituent, mean, unless otherwise stated, a fluorine, chlorine, bromine or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (e.g., from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

The term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen.

Each of the above terms (e.g., “alkyl,” “heteroalkyl,” “aryl” and “heteroaryl”) are meant to include both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, =0, =NR′, ′N—OR′, —NR′R″, —SW, -halo (or halogen), —SiR′R″R″t, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NRm, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the present application includes more than one R group, for example, each of the R groups is independently selected as are each R′, R″, R′″ and R″″ groups when more than one of these groups is present. Substituents on an aryl or heteroaryl groups are as provided for substituents on an alkyl group as defined above.

The term “acyl” describes a substituent containing a carbonyl residue, —C(O)R. Exemplary species for R include H, halogen, substituted or unsubstituted alkyl, substituted or unsubstituted aryl, substituted or unsubstituted heteroaryl, and substituted or unsubstituted heterocycloalkyl.

The term “fused ring system” means at least two rings, wherein each ring has at least 2 atoms in common with another ring. “Fused ring systems can include aromatic as well as non aromatic rings. Examples of “fused ring systems” are naphthalenes, indoles, quinolines, chromenes and the like.

The term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

The term “pharmaceutically acceptable salts” includes salts of the active compounds which are prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. When compounds of the present application contain relatively basic functionalities, acid addition salts can be obtained by contacting the neutral form of such compounds with a sufficient amount of the desired acid. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present application contain both basic and acidic functionalities that allow the compounds to be converted into either base or acid addition salts.

When a residue is defined as “O⁻”, then the formula is meant to optionally include an organic or inorganic cationic counterion. For example, the resulting salt form of the compound is pharmaceutically acceptable.

The neutral forms of the compounds are, for example, regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present application.

Certain compounds of the present application possess asymmetric carbon atoms (chiral centers) or double bonds; the racemates, diastereomers, geometric isomers and individual isomers are encompassed within the scope of the present application. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the present application can exist in particular geometric or stereoisomeric forms. The present application contemplates all such compounds, including cis- and trans-isomers, (−)- and (+)-enantiomers, diastereomers, (D)-isomers, (L)-isomers, the racemic mixtures thereof. All such isomers, as well as mixtures thereof, are intended to be included in this present application.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally includes a heteroatom. Thus, the term “5- to 7-membered ring” includes, for example pyridinyl and piperidinyl. The term “ring” further includes a ring system comprising more than one “ring”, wherein each “ring” is independently defined as above.

“Substituted or unsubstituted” or “optionally substituted” means that a group such as, for example, alkyl, aryl, heterocyclyl, (C₁₋₈)cycloalkyl, heterocyclyl(C₁₋₈)alkyl, aryl(C₁₋₈)alkyl, heteroaryl, heteroaryl(C₁₋₈)alkyl, and the like, unless specifically noted otherwise, may be unsubstituted or, may substituted by 1, 2 or 3 substituents selected from the group such as halo, nitro, trifluoromethyl, trifluoromethoxy, methoxy, carboxy, —NH₂, —OH, —SH, —NHCH₃, —N(CH₃)₂, —SMe, cyano and the like.

In one embodiment, there is provided aqueous compositions including a lipophilic bioactive molecule and a solubilizing agent described herein. In a particular aspect, the lipophilic bioactive molecule is ubiquinol ester or resveratrol ester. In another embodiment, the ubiquinol ester or resveratrol esters are the C₂₋₂₀ alkyl ester, C₂₋₁₀ alkyl ester, the C₂₋₆ alkyl ester, the C₂₋₅ alkyl ester, the C₂₋₃ alkyl ester or the C₂ alkyl ester.

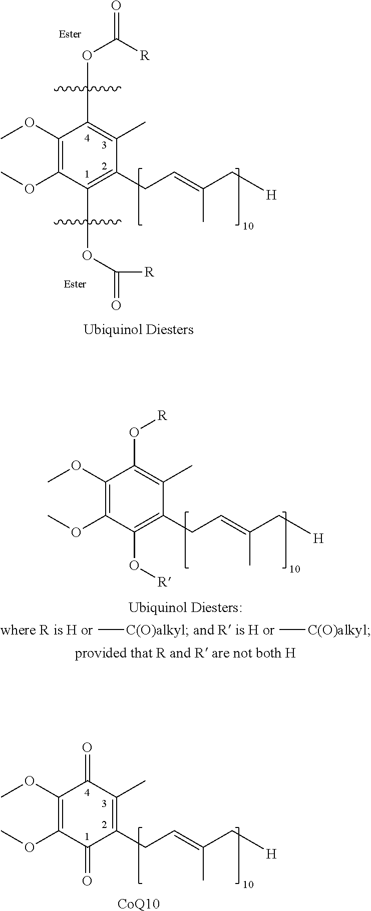

As used herein, a ubiquinol C₂ alkyl ester (where R is C₁ or —CH₃ in the above structure) may also be referred to as ubiquinol acetate, as the acetate portion (—OCOCH₃) of the ester group has 2 carbon atoms. In addition, the C₂ alkyl ester may also be referred to as a C₂ alkanoyl ester. Similarly, a ubiquinol C₃ alkyl ester (where R is C₂ or —CH₂CH₃) may also be referred to as ubiquinol propionate as the propionate portion of the ester group has 3 carbon atoms. In one embodiment, the diesters of ubiquinol are the same. In another embodiment, the two esters of ubiquinol are each independently C₂₋₁₀ alkyl esters.

In one embodiment, the composition comprising the molecules of the present application further comprises a mixture of ubiquinol ester/diester or resveratrol ester and at least a second lipophilic bioactive molecule. In one aspect, the second lipophilic bioactive molecule is ubiquinone or ubiquinone ester/diester. In another embodiment, the weight to weight (w/w) ratio of the ubiquinone to the ubiquinol ester/diester or resveratrol ester, used in the compositions of the present application is about 100:1, about 95:5, about 90:10, about 80:20, about 70:30, about 60:40, about 55:45, about 50:50, about 45:55, about 40:60, about 30:70, about 20:80, about 10:90, about 5:95 or about 1:100. In one embodiment of the above, the ubiquinol ester is the ubiquinol alkyl ester or di-alkyl ester, such as the C₂₋₂₀ alkyl ester, the C₂₋₁₀ alkyl ester, the C₂₋₆ alkyl ester or the C₂₋₅ alkyl ester or the C₂ alkyl ester. In a particular variation of the above, the ubiquinol ester or resveratrol ester is the acetate ester. In one variation, the ubiquinol ester/diester is the diacetate ester. In another variation, the resveratrol ester is a mono-ester, a diester or a tri-ester. In another variation of the above, the esters may be a C₂₋₂₀ alkyl ester, C₂₋₁₀ alkyl ester, C₂₋₆ alkyl ester, C₂₋₃ alkyl ester, C₂ alkyl ester or mixtures thereof.

These formulations have several advantages. First, they provide ubiquinol ester or resveratrol ester in an essentially clear, aqueous solution. This formulation can enable a consumer to ingest ubiquinol ester or resveratrol ester in a liquid form, for example, in a beverage, such as water. The aqueous formulations are essentially clear, which makes the formulations more appealing to a consumer. In another embodiment, the present application provides formulations (e.g., aqueous formulations) of ubiquinol ester or resveratrol ester that include a solubilizing agent described herein. The ubiquinol ester or resveratrol ester in these formulations (especially aqueous formulations) are stable with respect to chemical degradation (e.g., oxidation).

The present application also provides a method for making aqueous, water-soluble ubiquinol ester or resveratrol ester formulation of the present application.

In one example, the ubiquinol ester or resveratrol ester emulsion is formed using a solubilizing agent of the present application. In one example, the aqueous ubiquinol ester or resveratrol ester formulation thus formed is substantially clear. The water-soluble formulations of the present application may be used to prepare beverages having ubiquinol ester or resveratrol ester dissolved therein.

### Compositions:

The present application provides formulations of ubiquinol ester or resveratrol ester. In one another embodiment, the ubiquinol ester or resveratrol ester is the C₂₋₂₀ esters, the C₂₋₁₀ esters, the C₂₋₆ esters, the C₂-C₅ esters or the C₂ esters (or acetate).

In one embodiment, the formulations comprise at least (a) ubiquinol ester/diester or resveratrol ester, or mixtures thereof, as disclosed above, and (b) a solubilizing agent. In another embodiment, the formulations comprise at least (a) ubiquinol ester/diester or resveratrol ester or mixtures of bioactive molecules, as disclosed above and (b) a solubilizing agent. In one embodiment, the formulations comprise at least (a) a ubiquinol ester/diester or resveratrol ester of the present application and (b) a solubilizing agent. In another embodiment, the formulations comprise at least (a) a ubiquinol ester/diester or resveratrol ester of the present application, (b) a solubilizing agent and (c) a stabilizing agent. Stabilizing agents may be used in case the pH in the beverage causes some hydrolysis of the ubiquinol ester/diester, the presence of a stabilizing agent would prevent oxidation to yellow CoQ10.

The formulations of the present application can be used in a variety of products, such as foods, beverages, cosmetics and skin-care products (topical application), dietary supplements (e.g., formulated in soft-gelatine capsules) and nutraceuticals. In one embodiment, the present application provides a beverage including a formulation of the present application.

The following abbreviations are used throughout the application: TPGS—polyoxyethanyl-a-tocopheryl succinate (e.g., TPGS-1000). A number following one of the above abbreviations (e.g., TPGS-1000) indicates an average molecular weight of the polyoxyethanyl or poly(ethylene glycol) (PEG) moiety of the compound.

### Formulations:

In one embodiment, the present application provides a water-soluble formulation including ubiquinol ester or resveratrol ester or mixtures thereof as disclosed herein (e.g., a combination of ubiquinol ester or resveratrol ester), and a solubilizing agent of the present application. An alternative embodiment includes the above ingredients, but may rely on more than one solubilizing agent within any given formulation; (e.g., TPGS and other surfactants, in any ratio). In one aspect, the present application provides a water-soluble formulation including ubiquinol ester or resveratrol ester and a solubilizing agent of the present application. In one example, the solubilizing agent has a structure according to

Formula (I):

Y¹—[L¹]ₐ—Z (I)

wherein:

a is 0 and 1;

L¹ is a linker moiety that covalently links the hydrophobic moiety Z and the hydrophilic moiety Y¹;

Y¹ is a linear or branched hydrophilic linker moiety comprising at least one polymeric moiety independently selected from poly(alkylene oxides) (e.g., PEG) and polyalcohols, and monoethers; and

Z is a hydrophobic moiety.

In one aspect, Z is selected from the group consisting of sterols (e.g., cholesterol or sitosterol), tocopherols (e.g., alpha-tocopherol), tocotrienol and ubiquinol ester or resveratrol ester and derivatives or homologues thereof. In another aspect, the hydrophilic moiety is poly(ethylene glycol) (PEG) or methylated PEG (mPEG). In one example, L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene. In one embodiment, L¹ includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group. Exemplary functional groups according to this embodiment include ether, thioether, ester, carboxamide, sulfonamide, carbonate and urea groups. In a particular example, the solubilizing agent is polyoxyethanyl-a-tocopheryl succinate (TPGS).

In one aspect, Y¹ is a linear or branched hydrophilic moiety including at least one polymeric moiety, wherein each polymeric moiety is a member independently selected from poly(alkylene oxides) (e.g., PEG) and polyalcohols. Exemplary lipophilic moieties are described herein, each of which is useful in this embodiment. In one example, the lipophilic moiety is poly(ethylene glycol) (PEG) or methylated PEG (mPEG). In one embodiment, Y¹ is selected from poly(alkylene oxides) (i.e., polyethers), polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

PEG is usually a mixture of oligomers characterized by an average molecular weight. In one example, the PEG has an average molecular weight from about 200 to about 5000. In another aspect, PEG has an average molecular weight from about 500 to about 1500. In another aspect, PEG has an average molecular weight from about 500 to about 800 or about 900 to about 1200. In one example, the PEG is PEG-600 or is PEG-750. Both linear and branched PEG moieties can be used as the hydrophilic moiety of the solubilizing agent in the practice of the invention. In one aspect, PEG has between 1000 and 5000 subunits. In one aspect, the PEG is PEG 1000. In another aspect, PEG has between 100 and 500 subunits. In yet another aspect, PEG has between 10 and 50 subunits. In one aspect, PEG has between 1 and 25 subunits. In another aspect, PEG has between 15 and 25 subunits. PEG has between 5 and 100 subunits. In another aspect, PEG has between 1 and 500 subunits.

In one aspect, the ratio of the ubiquinol ester or resveratrol ester to the solubilizing agent is from about 1:0.1 (w/w), about 1:0.3, or a range of about 1:0.3 (w/w) to about 1:20 (w/w); or from about 1:1 (w/w) to about 1:20 (w/w), from about 1:1 (w/w) to about 1:10 (w/w); from about 1:1.3 (w/w) to about 1:5 (w/w), from about 1:2 (w/w) to about 1:4 (w/w), or is about 1:3 (w/w). In another variation, the ratio of the ubiquinol ester or resveratrol ester to the solubilizing agent is from about 1:0.1 (w/w) to about 1:0.3 (w/w), about 1:0.3 (w/w) to about 1:1 (w/w), or from about 1:0.5 (w/w) to about 1:2 (w/w).

### Water-Soluble Reducing Agent:

Certain compounds are known to be unstable toward oxidation. Microencapsulation using spray dry emulsions and complex coacervation technologies have been used to stabilize certain compounds for use in food products, but such methods do not provide stable aqueous formulations. C. J. Barrow et al, Lipid Technology, May 2007, Vol. 19, No. 5, 108-111.

In one example, according to any of the above embodiments, the formulation is an aqueous formulation and includes at least about 5% (w/w) of water, at least about 10%, at least about 20%, at least about 30%, at least about 40% or at least about 50% (w/w) of water. In another example, the aqueous formulation includes more than 50% (w/w) of water. For example, the aqueous formulation includes at least about 55%, at least about 60%, at least about 65%, at least about 70%, at least about 75% or at least about 80% (w/w) of water. In a further example, the aqueous formulation includes more than 80% (w/w) water. For example, the aqueous formulation includes at least about 85%, at least about 90%, at least about 92%, at least about 94% or at least about 96% (w/w) of water.

In one example, the ubiquinol ester or resveratrol ester are solubilized in the aqueous formulation through the formation of micelles that are generated most commonly by the self-aggregation of surfactant molecules, or alternatively, by inclusion of ubiquinol ester or resveratrol ester as part of the micelar array; i.e., mixed micelles formed between the ubiquinol ester or resveratrol ester and the solubilizing agent. The particle size of the formed micelles in solution may be measured using a dynamic light scattering (DLS) detector.

In one embodiment, the aqueous formulation of the present application is essentially clear (e.g., free of visible precipitation, cloudiness or haziness). In one example, the ubiquinol ester or resveratrol ester of the present application are formulated with TPGS resulting in an aqueous formulation that is essentially clear. Clear formulations of the present application can be colored. In one example, the formulation is essentially clear when the micelles have a particle size below about 150 nm. Hence, in another exemplary embodiment, the micelles formed by the solubilizing agent containing the ubiquinol ester or resveratrol ester have a median (average) particle size of less than about 100 nm. In another example, the micelles formed between the ubiquinol ester or resveratrol ester and the solubilizing agent, have a median particle size of less than about 90 nm, less than about 80 nm, less than about 70 nm or less than about 60 nm. In a further example, the micelles formed between the ubiquinol ester or resveratrol ester and the solubilizing agent, have a median particle size of less than about 50 nm, less than about 40 nm or less than about 30 nm. In another exemplary embodiment, the average particle size is from about 7 nm to about 90 nm. Another exemplary average particle size is from about 5 nm to about 70 nm, from about 10 nm to about 50 nm, from about 10 nm to about 30 nm, or from about 7 nm to about 10 nm. In a particular example, the micelles formed between the ubiquinol ester or the resveratrol ester and the solubilizing agent, have a median particle size between about 30 nm and about 10 nm (e.g., about 25 nm).

In another example, the aqueous formulation does not include an alcoholic solvent, although such inclusion is possible when part of the solubilizing agent (e.g., as in Cremophore, which contains ethanol). Exemplary alcoholic solvents include solvents, such as ethanol, methanol, propanol, butanol and higher alcohols (e.g., C₅-C₂₀ alcohols). Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of ubiquinol ester or resveratrol ester in the formulation is at least about 20 mg/mL and can be as high as about 60, about 80, about 100 or more than about 100 mg/mL. In one example, the concentration of ubiquinol ester or resveratrol ester in the aqueous formulation of the present application is at least about 1 mg/mL, at least about 5 mg/mL, at least about 10 mg/mL, at least about 20 mg/mL, at least about 30 mg/mL, at least about 40 mg/mL, at least about 50 mg/mL, at least about 60 mg/mL, at least about 70 mg/mL or at least about 80 mg/mL, at least about 85 mg/mL, at least about 90 mg/mL, at least about 95 mg/mL or at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL or at least about 200 mg/mL. In another example, the concentration of ubiquinol ester or resveratrol ester in the aqueous formulation is greater than 200 mg/mL.

In one embodiment, the present application provides a water-soluble formulation comprising ubiquinol ester or resveratrol ester, a water-soluble reducing and/or antioxidizing agent, water-insoluble reducing and/or antioxidizing agent and a solubilizing agent. In another embodiment, the present application provides a water-soluble formulation comprising ubiquinol ester or resveratrol ester, a water-soluble antioxidant and a solubilizing agent. In one example, the solubilizing agent has a structure according to Formula (I) described herein.

In particular variations of each of the above aspects and embodiments, the formulation may comprise ubiquinol ester or resveratrol ester and TPGS-1000; natural, non-natural and synthetic surfactants and mixtures of surfactants, including, for example, two or more surfactants of differing structural types (e.g., TPGS-1000 and Tween-80), two or more surfactants from within the same structural class (e.g., TPGS-1000). In another variation of the above formulations, the formulations may also comprise any of the above combinations as their free alcohols, or as their ether or ester derivatives (of their PEG portion). In another particular variation of the above formulations, the formulations may also comprise antioxidants that are lipophilic in nature (e.g., vitamin C palmitate), hydrophilic in nature (e.g., vitamin C), and any combinations of these, including more than one of each in any formulations. In another particular variation of the above formulations, the formulations may also comprise chelating agents that are lipophilic in nature, hydrophilic in nature (e.g., EDTA, HEDTA, DTPA and NTA), and any combinations of these, and in any number (i.e., more than one of each in any formulation) or ratio. In another particular variation of the above, the formulations may also comprise salts such as salts that are lipophilic in nature (e.g., ammonium salts, such as R₄N⁺X⁻), hydrophilic in nature (e.g., sodium benzoate), and/or a stabilizing agent (e.g., potassium sorbate), where any combinations of these, and in any number (i.e., more than one of each in any formulation) or ratio, may vary with each application. According to the present formulations, variations of each of ubiquinol ester or resveratrol ester, the surfactants, the antioxidants, lipophilic and hydrophilic salts, and each of these elements and their combinations, may be used to provide the stable, water soluble bioactive agents such as the ubiquinol ester or resveratrol ester formulations of the present application.

In one example, the ubiquinol esters or resveratrol esters are emulsified in the formulation in the form of micelles that include the ubiquinol ester or resveratrol ester and the solubilizing agent. In a typical emulsion of the present application, the micelles are small in size, and are between about 10 and about 100 nm. In another example, the small size of the micelles causes the emulsion to be essentially clear in appearance even at high compound concentrations (e.g., 40, 60, 80 or 100 mg/mL). In one example, the ubiquinol ester/diester or resveratrol ester concentration in the aqueous formulations of the present application is at least about 20 mg/mL and can be as high as about 60, about 80, about 100 or more than about 100 mg/mL.

### Beverages:

In another example, the present application provides a mixture between a formulation of the present application (e.g., a water-soluble formulation) and an original beverage to create a beverage of the present application. The original beverage can be any beverage (e.g., a clear beverage). Exemplary original beverages are described herein and include carbonated or non-carbonated waters, flavored waters, soft drinks and the like. In one example, the mixture (beverage of the present application) includes between about 1 mg/L and about 1000 mg/L of solubilized ubiquinol ester or resveratrol ester. In another example, the mixture includes between about 10 mg/L and about 500 mg/L of solubilized ubiquinol ester or resveratrol ester, between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinol ester or resveratrol ester. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized ubiquinol ester or resveratrol ester. In one aspect, the beverage may comprise of about 1,000 mg or less of solubilized ubiquinol ester or resveratrol ester. In another aspect, the beverage may comprise of about 500 mg or less of solubilized ubiquinol ester or resveratrol ester. In another aspect, the beverage may comprise of about 250 mg or less of solubilized ubiquinol ester or resveratrol ester. In one aspect, the beverage may comprise of a range of about 10 mg to about 500 mg per serving. In another aspect, the beverage may comprise of a range of about 25 mg to about 500 mg per serving. In certain aspects, the beverage may have two servings. In certain variation of the beverage, the beverage may comprise about 15% to about 30% of the daily recommended value of the ubiquinol ester or resveratrol ester.

In one embodiment, the concentration of the ubiquinol ester or resveratrol ester in the formulation provides the daily recommended dose for CoQ10. In one aspect, the formulation provides up to about 500 mg of CoQ10 per serving.

In a particular example according to any of the above embodiments, the present application provides a mixture between ubiquinol ester or resveratrol ester formulation of the present application and an original beverage (e.g., carbonated or non-carbonated water) to form a ubiquinol ester or resveratrol ester beverage. In another aspect, the present application provides a non-alcoholic beverage comprising (a) solubilized ubiquinol ester or resveratrol ester and (b) a solubilizing agent.

In another embodiment, the ubiquinol ester or resveratrol ester beverage contains between about 1 mg/L and about 1000 mg/L of solubilized ubiquinol ester or resveratrol ester, between about 10 mg/L and about 500 mg/L of solubilized ubiquinol ester or resveratrol ester, between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinol ester or resveratrol ester. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized ubiquinol ester or resveratrol ester.

In another example, according to any of the above embodiments, the beverage further includes a coloring agent and/or a flavoring agent. It is possible to add one or more fruit and/or vegetable juice concentrates and/or flavor improvers to the beverage. For example, a mixture of about LIMETTE citrus (e.g., about 1.38 g/l), cassis (e.g., about 1.04 g/l), mango (e.g., about 1.04 g/l) or combinations thereof, can be added to the beverage. In another example, maltodextrin (e.g., about 20 g/l), fructose (e.g., about 50 g/l) or combinations thereof can be added to the beverage. In another example, the finished beverage is subjected to a primary and, optionally, a secondary filtration.

In yet another example according to any of the above embodiments, the ubiquinol ester/diester or resveratrol ester is stabilized and solubilized in the beverage. For example, the beverage is essentially free of CoQ10 or ubiquinol ester, or resveratrol or resveratrol ester precipitation. In another example, the beverage is essentially clear. Clarity of a beverage can be assessed using turbidity measurements. In one example, the turbidity of the ubiquinol ester or resveratrol ester beverage is comparable (e.g., not more than 5 times) of the turbidity of the control beverage. In one example, the turbidity of the ubiquinol ester or resveratrol ester beverage is not more than about 500%, not more than about 400%, not more than about 300% or not more than about 200% higher than the turbidity of the control. In yet another example, the turbidity is not more than about 180%, not more than about 160%, not more than about 140%, not more than about 120% or not more than about 100% higher than the turbidity of the control. The turbidity is 100% higher than the control, when the turbidity of the beverage is twice as high as the turbidity of the control.

In addition, the beverage comprising the formulation or composition of the present application can be enriched with vitamins. In one example, the beverage includes at least one B vitamin. Exemplary B-vitamins include vitamin B1, vitamin B2, vitamin B3 and vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the ubiquinol ester or resveratrol ester.

In one example, the formulation includes from about 0.01% (w/w) to about 0.1% (w/w) of an ubiquinol ester or resveratrol ester, from about 0.01% (w/w) to about 0.5% (w/w), from about 0.01% (w/w) to about 1% (w/w), from about 0.05% (w/w) to about 0.25% (w/w), from about 0.1% (w/w) to about 1% (w/w), from about 0.1% (w/w) to about 0.75% (w/w), from about 1% (w/w) to about 3% (w/w), from about 1% (w/w) to about 10% (w/w), from about 1% (w/w) to about 20% (w/w), from about 1% (w/w) to about 30% (w/w), from about 1% (w/w) to about 40% (w/w), from about 5% to about 50% by weight, or from about 10% to about 30% (w/w), for example, from about 15% to about 25% (w/w).

### Solubilizing Agents Wherein Z is a Tocopherol or a Tocotrienol:

In another embodiment, Z is selected from a substituted or unsubstituted tocopherol and a substituted or unsubstituted tocotrienol. In one example, Z is an α-, β-, γ-, or δ-tocopherol. α-(+)-Tocopherol (natural) and α-(±)-tocopherol (synthetic) are preferred tocopherols, with synthetic racemic tocopherol being particularly preferred for TPGS. In another embodiment, Z has a structure according to the following formula:

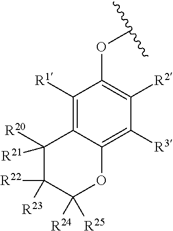

wherein R¹′, R²′ and R³′ are independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R²′ and R³′, together with the carbon atoms to which they are attached, are optionally joined to form a 5- to 7-membered ring. R²⁰, R²¹, R²², R²³, R²⁴ and R²⁵ are selected from H, halogen, nitro, cyano, OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In another embodiment, at least one of R²⁴ and R²⁵ comprises an isoprene moiety. In another embodiment, R¹′, R²′ and R³′ are independently selected from H and methyl. In another exemplary embodiment, R³′ is methyl, R²′ is methyl and R¹′ is methyl. In another exemplary embodiment, R³′ is methyl, R²′ is H and R¹′ is methyl. In another exemplary embodiment, R³′ is methyl, R²′ is methyl and R¹′ is H. In another exemplary embodiment, R³′ is methyl, R²′ is H and R¹′ is H.

In one example, Z has a structure according to the following formulae:

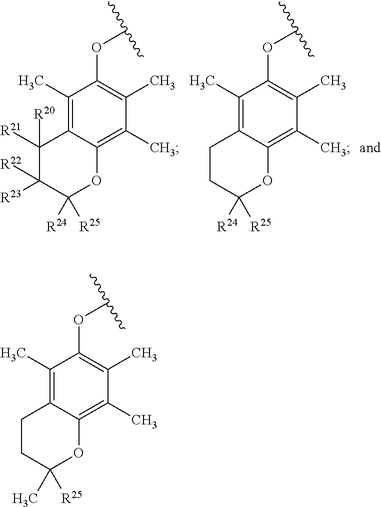

wherein R²⁷ is selected from substituted or unsubstituted alkyl and substituted or unsubstituted heteroalkyl. In one example, R²⁴ is methyl. In another example, R²⁵ includes a moiety having a structure selected from the following formulae:

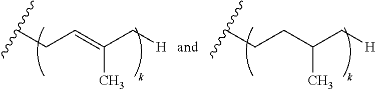

wherein k is an integer selected from 1 to 12. In another embodiment, k is from 2 to 6. In another exemplary embodiment, k is 3.

In another embodiment, the solubilizing agent has a structure according to the following formula:

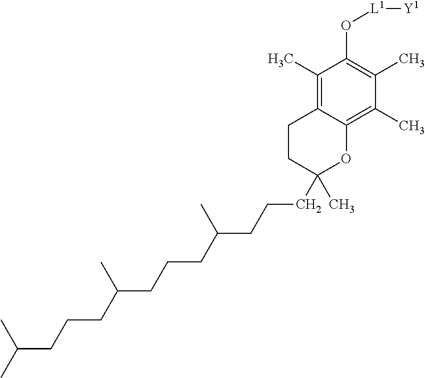

In another embodiment, the moiety L¹-Y¹ has a structure according to the following formula:

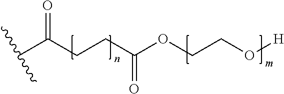

wherein n is selected from 1 to 20, m is selected from 1 to 5000. In another embodiment, n is 4. In another embodiment, m is a selected from 1 to 2,500.

Methods of making the above solubilizing agents are known in the art. For example, the methods of making certain solubilizing agents are disclosed in U.S. Pat. Nos. 6,045,826, 6,191,172, 6,632,443 and WO 96/17626, all herein incorporated by reference in their entirety. Similarly, TPGS may be prepared accordingly, or by using succinic anhydride as the linker in place of the diacid chloride as precursor to the four-carbon linker.

### Specific Sterols and Linkers:

In another embodiment, the solubilizing agent has a structure, which is a member selected from:

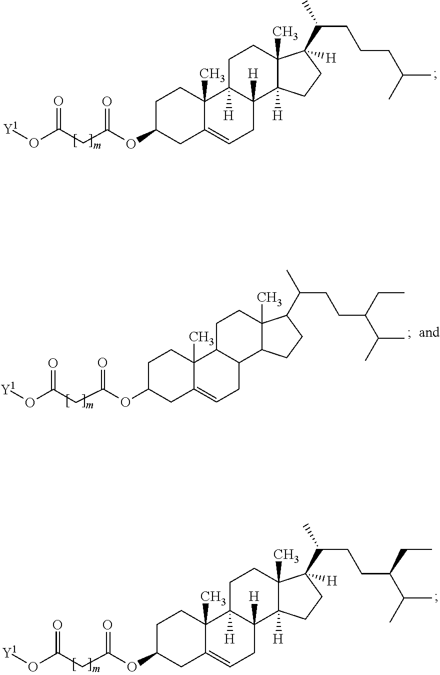

wherein m is selected from 2-16. In one example, m is selected from 2, 6, 8, 10, 12 and 14. In another example, m is 2. In yet another example, m is 8.

### Specific Sterols and PEG:

In another embodiment, the solubilizing agent is selected from

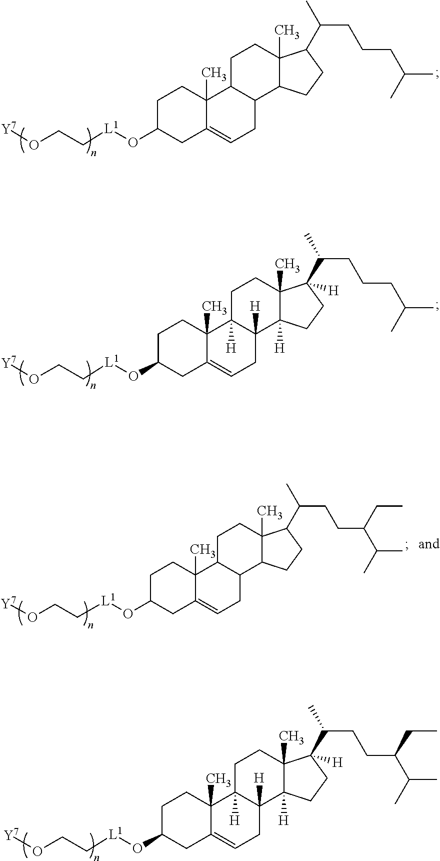

wherein n is selected from 10 to 2500, L¹ is a linker moiety, Y⁷ is selected from H and methyl.

### Specific Tocopherols and Linkers:

In another embodiment, the solubilizing agent has a structure according to one of the following formulae:

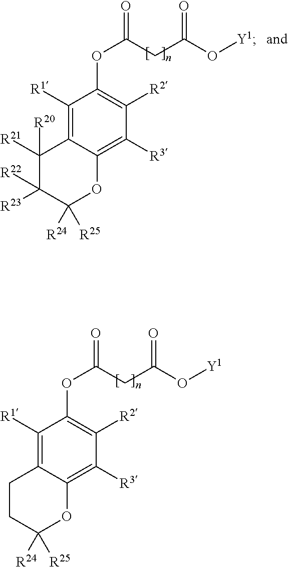

wherein n is an integer selected from 1 to 20. Y¹, R¹′, R²⁰, R²¹, R²², R²³, R²⁴ and R²⁵ are defined as herein above.

### Specific Tocopherols and PEG:

In another embodiment, the solubilizing agent has a structure according to the following formula:

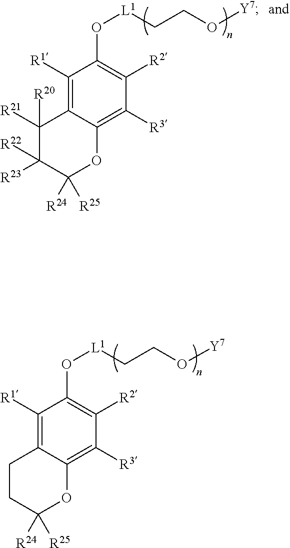

wherein n is a member selected from 10 to 2500. L¹, R¹′, R²′, R³′, R²⁰, R²¹, R²², R²³, R²⁴ and R²⁵ are defined as herein above. Y⁷ is selected from H and methyl.

In another embodiment, the formulations of the present application include from about 10% to about 50% by weight of a solubilizing agent. The formulations include from about 15% to about 40% (w/w) solubilizing agent, from about 20% to about 40% (w/w), and from about 20 to about 35% (w/w). In another embodiment, the present application includes from about 0.01% (w/w) to about 5% (w/w), from about 0.01% (w/w) to about 0.1% (w/w), from about 0.01% (w/w) to about 1% (w/w), from about 0.1% (w/w) to about 1% (w/w), from about 0.1% (w/w) to about 0.75% (w/w), 1% (w/w) to about 3% (w/w), and from about 0.05% (w/w) to about 0.25% (w/w) of a solubilizing agent.

In another embodiment, the stabilizer is in excess in relation to the ubiquinol ester or resveratrol ester. In another exemplary embodiment, the ratio of the ubiquinol ester or resveratrol ester to the stabilizer is from about 1:1 (w/w) to about 1:6 (w/w), from about 1:1 (w/w) to about 1:5 (w/w), from about 1:1.3 (w/w) to about 1:3 (w/w), from about 1:2 (w/w) to about 1:4 (w/w), or about 1:3 (w/w).

In another embodiment, the stabilizer is vitamin C or a vitamin C derivative. In one example, the vitamin C or the vitamin C derivative is used in a molar excess in relation to the lipophilic bioactive molecule. In another exemplary embodiment, the ratio of the lipophilic bioactive molecule to said vitamin C or vitamin C derivative is from about 1:1 (w/w) to about 1:6 (w/w), from about 1:1 (w/w) to about 1:10 (w/w), from about 1:1.3 (w/w) to about 1:5 (w/w), from about 1:2 (w/w) to about 1:4 (w/w), or about 1:3 (w/w).

### Other Components:

The formulations described herein (either aqueous or non-aqueous) can further include various ingredients useful to stabilize the composition, promote the bioavailability of the lipophilic bioactive molecule, or provide nutritional value. Exemplary additives of the present formulations include, without limitation, one or more alternative solubilizing agents, pharmaceutical drug molecules, antibiotics, sterols, vitamins, provitamins, carotenoids (e.g., alpha and beta-carotenes, cryptoxanthin, lutein and zeaxanthin), phospholipids, L-carnitine, starches, sugars, fats, stabilizers, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

Vitamin(s) in a unit dosage form of the present application are present in amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s) in a unit dosage form of the present application are present in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg. In the formulations of the present application the additional components are usually a minor component (from about 0.001% to about 20% by weight or preferably from about 0.01% to about 10% by weight) with the remainder being various vehicles or carriers and processing aids helpful for forming the desired dosing form.

### Pharmaceutical Formulations:

### Exemplary Formulations Including Stabilizers:

In another embodiment, the present application provides a formulation which comprises: (a) a ubiquinol ester or resveratrol ester; (b) a solubilizing agent; and (c) EDTA. In another embodiment, the ratio of the ubiquinol ester or resveratrol ester to said solubilizing agent is from about 1:0.3 (w/w) to about 1:20 (w/w), from about 1:1 (w/w) to about 1:20 (w/w), from about 1:1 (w/w) to about 1:10 (w/w), from about 1:1.3 (w/w) to about 1:5 (w/w), from about 1:2 (w/w) to about 1:4 (w/w), about 1:3 (w/w); from about 1:0.3 (w/w) to about 1:1 (w/w), or from about 1:0.5 (w/w) to about 1:2 (w/w). In another embodiment, the ratio of the ubiquinol ester or resveratrol ester to TPGS is from about 1:2 to about 1:4, or about 1:3.

In another embodiment, the present application provides a formulation which comprises: (a) a ubiquinol ester or resveratrol ester; (b) a solubilizing agent; (c) vitamin C, a vitamin C derivative, or combinations thereof; and (d) EDTA. In one embodiment, the ubiquinol ester or resveratrol ester is present in the formulation in an amount of at least about 0.5% by weight, at least about 1% by weight, at least about 1.5% by weight, at least about 2% by weight, at least about 2.5% by weight, at least about 3% by weight, at least about 3.5% by weight, at least about 4% by weight, at least about 4.5% by weight or at least about 5% by weight. In another embodiment, the ubiquinol ester or resveratrol ester is present in the formulation in an amount of at least about 95% by weight, at least about 96% by weight or at least about 97% by weight.

In another embodiment, the present application provides a formulation which comprises: (a) ubiquinol ester or resveratrol ester; (b) a solubilizing agent; (c) a stabilizer; and (d) EDTA. In another embodiment, the present application provides a formulation which comprises: (a) ubiquinol ester or resveratrol ester; (b) a solubilizing agent; (c) Vitamin C, a Vitamin C derivative, or combinations thereof; and (d) EDTA. In another embodiment, the present application provides a formulation which comprises: (a) ubiquinol ester or resveratrol ester; (b) TPGS; (c) Vitamin C, a Vitamin C derivative, or combinations thereof; and (d) EDTA. In another embodiment, the present application provides a formulation which comprises: (a) ubiquinol ester or resveratrol ester; (b) a solubilizing agent; (c) a stabilizer; and (d) EDTA. In another embodiment, the present application provides a formulation which comprises: (a) ubiquinol ester or resveratrol ester; (b) a solubilizing agent; (c) Vitamin C, a Vitamin C derivative, or combinations thereof.

### Cosmetic or Topical Formulations:

The composition of the present application may also include pharmaceutical carriers that may be topically applied onto the skin or mucosal membranes. In one aspect, ointments containing a hydrophobic carrier or petroleum as a carrier may be applied onto a skin surface. In another aspect, the formulation may include a hydrophobic carrier such as petroleum. Hydrophobic pharmaceutical carriers may include liquid paraffin, lanolin, beeswax, vegetable oil, glycerin monostearate, higher molecular weight alcohols, polyethylene glycol and optionally, emulsifying agents. The formulations may also include hydrophobic liquids, such as mono- and poly-unsaturated oils, such as vegetable oils or marine based oils, silicone oils, mineral oils, and liquid hydrophobic plant-derived oils. In one aspect, the oils may also contain essential nutritional constituents, such as oil-soluble vitamins (e.g., vitamin A and vitamin E), minerals and other therapeutically effective constituents. Other therapeutic oils may include mineral oil and silicone oil.

### Methods:

### Methods of Making the Formulations:

The present application also provides methods (e.g., processes) of making the formulations and compositions of the present application. In one embodiment, the ubiquinol ester or resveratrol ester, a solubilizing agent and EDTA, and optionally other components of the formulation are placed in a container. A solvent is then added and the mixture is optionally heated, thereby dissolving the components and forming the formulation. In another exemplary embodiment, the ubiquinol ester or resveratrol ester is dissolved in a solvent optionally using heat. The solubilizing agent, and optionally other components are added to the above solution creating a mixture, which is stirred and optionally heated to dissolve all components in the mixture, thus creating the formulation. In another embodiment, a solubilizing agent is dissolved in a solvent (e.g., water). The ubiquinol ester or resveratrol ester and EDTA, together with any optional components are added and dissolved in the above solution (optionally using heat), thus creating the formulation. In another exemplary embodiment is dissolved in a solvent of choice. The ubiquinol ester or resveratrol ester and the solubilizing agent and EDTA, together with any optional components are added and are dissolved in the solution (optionally using heat), thus creating the formulation.

### Exemplary Processes:

In a particular example, the solubilizing agent is polyoxyethanyl-a-tocopheryl succinate (TPGS). In one example, the ubiquinol ester or resveratrol ester is solubilized in the above emulsion in the form of micelles that are formed between the ubiquinol ester or resveratrol ester and the solubilizing agent. In one example, the micelles have a median particle size of less than about 60 nm (e.g., between about 10 and about 30 nm). In one example, the present application provides a ubiquinol ester or resveratrol ester stock solution, which is prepared by a method according to any of the above embodiments. In one example, the above water-soluble ubiquinol ester or resveratrol ester stock solution can be used to prepare a beverage of the present application. In one embodiment, the above method further includes contacting the water-soluble ubiquinol ester or resveratrol ester stock solution with an original beverage to form a ubiquinol ester or resveratrol ester beverage of the present application. Exemplary original beverages useful in the methods of the present application are disclosed herein.

### Methods of Making the Beverages:

In another aspect, the present application provides a method for making a beverage (e.g., a non-alcoholic beverage) that includes ubiquinol ester or resveratrol ester. An exemplary method includes: contacting an original beverage with a water-soluble ubiquinol ester or resveratrol ester stock solution (e.g., ubiquinol ester or resveratrol ester-50 stock solution) of the present application. Exemplary original beverages are disclosed herein and include carbonated or uncarbonated water, flavored water, soft drinks, beer and drinkable dairy products.

All embodiments described herein above for the method of making a ubiquinol ester or resveratrol ester stock solution equally apply to the method of making a ubiquinol ester or resveratrol ester beverage described in this paragraph. In one example, the method further includes adding a vitamin (e.g., vitamin C, vitamin E, a B-vitamin (e.g., vitamin B-pentapalmitate) or combinations thereof) to the beverage. In one example, when the vitamin (e.g., vitamin E) is added to the beverage, the vitamin is first solubilized in an aqueous medium using a solubilizing agent, such as a solubilizing agent of the present application, and is subsequently added to the beverage. Exemplary solubilizing agents that can be used to solubilize the vitamin (e.g., vitamin E) include TPGS and polyoxyethylene sorbitan monooleate. In another embodiment, the present application provides a beverage produced by any of the above methods of the present application.

The compositions and methods of the present application are further illustrated by the examples that follow. These examples are offered to illustrate, but not to limit the claimed present application.

### METHODS AND PROCEDURES

As provided herein, the present application provides a method for preparing clear and stable ubiquinol ester or resveratrol ester compositions for use in various food products. In a particular embodiment, the ubiquinol ester or resveratrol ester formulations described herein are nano-emulsions. As disclosed herein, the composition is safe for human consumption. The compositions are GRAS, self-affirmed GRAS (TPGS-1000), or the composition comprises other food materials. In addition, the composition that are used are inexpensive, readily sourced from bulk materials, and may cost less than about $100/kg.

In one aspect, the ratio of the surfactant to the ubiquinol ester or resveratrol ester is low, such as a ratio of less than 2:1 or about 1:1(w/w). The compositions prepared from the methods described herein are relatively clear at concentration and dilution. The compositions prepared by the present methods provide NTU levels that are in the low double digit numbers, however, in certain formulations, the composition may provide compositions as high as 200 NTU.

In certain embodiments, the formulations comprise a high percentage of the daily allowable dose of the emulsion ingredient such that the ubiquinol ester or resveratrol esters are provided in high delivery dosages. In a particular aspect, the emulsifier that is present does not present a significant taste and odor profile. In certain embodiments, the surfactants employed in the present application may include:

Hydrophilic Lipophilic Balance (HLB)>10 such as Poloxamer 188, Polysorbate 80, Polysorbate 20, Vitamin E-TPGS, Solutol HS 15, PEG-40, Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14) and PEG-32-glyceryl palmitostearate (Gelucire 50/13).

HLB 8-12 such as Polysorbate 85, polyglyceryl-6-dioleate (Caprol MPGO), TPGS, and/or mixtures of high and low HLB emulsifiers.

HLB<8 such as sorbitan monooleate (Span 80), Capmul MCM, maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), propylene glycol monolaurate (e.g., Capmul PG-12 or lauroglycol 90), polyglyceryl-3 dioleate (Plurol Oleique CC497), polyglyceryl-3 diisostearate (Plurol Diisostearique) and lecithin with and without bile salts.

The relative solubility of compositions of the present application, including composition comprising, for example, a 2:1 and 1:1 surfactant/ubiquinol ester or resveratrol ester systems in water (or other aqueous solvent system(s)) may be determined by emulsification screening, visual appearance, turbidity, tarticle (emulsion droplet) size by Photon Correlation Spectroscopy (PCS), visual assessment of dilution effects, ambient room temperature (RT) stability at 1, 2 and 4 weeks and established compatibility with beverage matrices. Certain surfactants include the tweens, such as tween 60 and tween 65, tweens 80, 96, 99, 89. Suitable antioxidants and stabilizers may be added at the desired concentrations to provide the desired compositions.

As provided herein, the compositions of the present application demonstrate significant oxidative stability, and may be tested and determined by storing the composition in vials. The composition may be purged with oxygen and analyzed at various time intervals to determine compositions having the optimal appearance, the assay (by HPLC, for example), by PCS and the physical and chemical stability suitable for use in various food products.

### EXAMPLES

Into a 12 Liter round bottomed flask, equipped with a thermometer, an overhead stirrer and a heating mantle, under a blanket of nitrogen gas is added TPGS-600 (1.32 moles). Ascorbic acid (12.0 grams, 0.07 moles) is added to the flask. Ethylenediaminetetraacetic acid disodium salt dihydrate (35 grams, 0.09 moles) is added to the flask. MTD10 (70 grams) is then added to the flask. L-Ascorbic acid-6-palmitate (70.0 grams, 0.17 moles) is then added to the flask. Water (5,600 grams, 311.1 moles) is then charged to the flask, and the resulting stirred reaction mixture is heated to 90 to 95° C. and maintained at about 90 to 95° C. for two hours.

Ubiquinol ester/diester or resveratrol ester (about 300 grams) is charged to the heated flaks via cannula under nitrogen, and the resulting mixture is stirred at 90 to 95° C. for 30 minutes. The mixture then turned into an emulsion. The reaction mixture is cooled to about 10-15° C. at a rate of greater than about 10° C. per hour using a temperature controller.

Once the reaction mixture is clear, a sample is obtained from the reaction flask and analyzed. IPC is determined.

Solubilization of High Grade Ubiquinol Ester or Resveratrol Ester with TPGS:

In a microcentrifuge tube, ubiquinol ester or resveratrol ester (100 mg), Vitamin E TPGS (200 mg, Antares) is combined and heated until a melt is obtained. Then DI water (700 mg) is added and the mixture is heated to 90-100° C. until it became homogeneous. The homogeneous mixture is cooled to room temperature and is slightly opaque. The opaque homogeneous mixture stayed in solution for ˜4 days. Then 64.5 mg of the opaque homogeneous mixture is diluted with 30 mL of DI water and the clarity is measured at 7.97 NTU.

In a microcentrifuge tube, ubiquinol ester or resveratrol ester (100 mg), Vitamin E TPGS (200 mg, Antares) and DI water (700 mg) are combined. The mixture is heated to 90-100° C. until it became homogeneous. The homogeneous mixture is cooled in and ice bath and is opaque. After ˜5 days the once opaque homogeneous mixture is observed to be heterogeneous. The mixture is reheated to 90-100° C. until it became homogeneous. The homogeneous mixture is cooled in and ice bath and is an opaque homogeneous mixture. Then 62 mg of the opaque homogeneous mixture is diluted with 30 mL of DI water and the clarity is measured at 14.9 NTU.

In a microcentrifuge tube, ubiquinol ester or resveratrol ester (50 mg), Vitamin E TPGS (300 mg, Antares) is combined and heated until a melt is obtained. Then DI water (1050 mg) is added and the mixture is heated to 90-100° C. until it became homogeneous. The homogeneous mixture is cooled in and ice bath and is an opaque homogeneous mixture. Then all of the mixture is diluted with 30 mL of DI water and the clarity is measured at 48.1 NTU.

In a 250 mL 3-neck round bottom flask equipped with an overhead stirrer, thermocouple, condenser, heating mantle and a nitrogen inlet, ubiquinol ester or resveratrol ester (10.2 g), Vitamin E TPGS (20 g, Antares) and DI water (70 g) are combined. The mixture is heated to 95° C. until it became homogeneous, and is held for 30 minutes. Then the homogeneous mixture is cooled in an ice water bath. At 85° C. the opaque homogeneous mixture became a clear homogeneous mixture. After further cooling to 2.8° C., the clarity is measured at 99.2 NTU.

In a 250 mL 3-neck round bottom flask equipped with an overhead stirrer, thermocouple, condenser, heating mantle and a nitrogen inlet, Vitamin E TPGS (20.0 g, TR Nutritionals), ubiquinol ester/diester or resveratrol ester (10.0 g), and DI water (70.0 g) are combined. The mixture is heated to 95° C. until it became homogeneous, and is held for ˜45 minutes. Then the opaque homogeneous mixture is cooled in an ice water bath. After cooling to 5° C., the mixture remained homogeneous but is opaque. Then 61.1 mg of this solution is diluted with 30 mL of DI water and the clarity is measured at 72.1 NTU.

Solubility, clarity and stability results of the solution prepared according to the procedures as described herein demonstrate that the formulations as described herein maintains clarity and stability for the desired period of time under the storage conditions.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A composition comprising:
a) a ubiquinol ester/diester or a resveratrol ester, or a mixture thereof; and
b) a solubilizing agent comprising the Formula (I):
Y¹—[L¹]ₐ—Z (I)
wherein:
a is 0 and 1;
L¹ is a linker moiety that covalently links the hydrophobic moiety Z and the hydrophilic moiety Y¹;
Y¹ is a linear or branched hydrophilic moiety comprising at least one polymeric moiety independently selected from poly(alkylene oxides) and polyalcohols; and
Z is a hydrophobic moiety.

2. The composition of claim 1, wherein the ubiquinol ester/diester or the resveratrol ester is selected from the group consisting of amino acid esters, nutritional acid esters, C₂₋₂₀ alkyl ester and C₆₋₂₀ aryl ester, and mixtures thereof.

3. The composition of claim 2, wherein the ubiquinol ester/diester or the resveratrol ester is selected from the group consisting of the C₂₋₁₂ alkyl ester, the C₂₋₁₀ alkyl ester, the C₂₋₅ alkyl ester and the C₂-C₃ alkyl ester, and mixtures thereof.

4. The composition of claim 2, wherein the nutritional acid esters are selected from the group consisting of omega-3, omega-6, and omega-9 fatty acids, α-linolenic acid (ALA), stearidonic acid, eicosatetraenoic acid, eicosapentaenoic acid (EPA), docosapentaenoic acid, docosahexaenoic acid (DHA), linoleic acid, gamma-linolenic acid, eicosadienoic acid, dihomo-gamma-linolenic acid, arachidonic acid, docosadienoic acid, adrenic acid, docosapentaenoic acid, oleic acid, eicosenoic acid, mead acid, erucic acid, nervonic acid, vitamin B, vitamin B-3, biotin, folic acid, pantothenic acid, para-amino benzoic acid and taurine, and mixtures thereof.

5. The composition of claim 1, wherein the composition further comprises water to form an aqueous composition, wherein the aqueous composition is a substantially clear, water soluble composition.

6. The composition of claim 1, wherein the solubilizing agent comprises the Formula (I), wherein:
Z is selected from the group consisting of sterols, tocopherols, tocotrienol and ubiquinol ester/diester and derivatives or homologues thereof;
L¹ is selected from a single bond, substituted or unsubstituted alkylene, substituted or unsubstituted heteroalkylene, substituted or unsubstituted arylene, substituted or unsubstituted heteroarylene and substituted or unsubstituted heterocycloalkylene; and
Y¹ is a linear or branched hydrophilic moiety including at least one polymeric moiety, wherein each polymeric moiety is a member independently selected from poly(alkylene oxides) and polyalcohols.

7. The composition of claim 6, wherein:
Y¹ is selected from the group consisting of poly(alkylene oxides) and monoethers therefrom, polyalcohols, polysaccharides, polyamino acids, polyphosphoric acids, polyamines and derivatives thereof; and
L¹ is selected from the group consisting of a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkylene chain, optionally incorporating at least one functional group selected from the group consisting of ether, thioether, ester, carboxamide, sulfonamide, carbonate and urea groups.

8. The composition of claim 1, wherein the solubilizing agent is TPGS (polyoxyethanyl-a-tocopheryl succinate) or TPGS-1000 (polyoxyethanyl-a-tocopheryl succinate-1000).

9. The composition of claim 1, further comprising a stabilizing agent.

10. A method for stabilizing a bioactive compound selected from the group consisting of ubiquinol ester/diester and resveratrol ester, and mixtures thereof, in an aqueous solution comprising contacting the bioactive compound, with a composition comprising a micelle-forming surfactant for a sufficient period of time to dissolve the bioactive compound.

11. The method of claim 10, wherein the bioactive compound is selected from the group consisting of a ubiquinol ester/diester and resveratrol ester selected from the group consisting of the C₂₋₂₀ alkyl ester, the C₂₋₁₀ alkyl ester, the C₂₋₆ alkyl ester or the C₂₋₃ alkyl ester, and mixtures thereof.

12. The method of claim 10, wherein the micelle-forming surfactant is TPGS (polyoxyethanyl-a-tocopheryl succinate) or TPGS-1000 (polyoxyethanyl-a-tocopheryl succinate-1000).

13. The method of claim 10, wherein the composition further comprises a stabilizing agent.

14. The method of claim 13, wherein contacting the bioactive compound with the composition comprising a micelle-forming surfactant for a sufficient period of time to dissolve the bioactive compound is performed at an elevated temperature.

15. A method for increasing the bioavailability and/or the absorption of ubiquinol or resveratrol, or mixtures thereof, in a mammal, comprising:
preparing an absorption enhanced bioactive formulation of a ubiquinol ester/diester or a resveratrol ester, wherein the bioactive formulation comprises:
a) a ubiquinol ester/diester or a resveratrol ester, or mixtures thereof; and
b) a solubilizing agent comprising the Formula (I):
Y¹—[L¹]ₐ—Z (I)
wherein:
a is 0 and 1;
L¹ is a linker moiety that covalently links the hydrophobic moiety Z and the hydrophilic moiety Y¹;
Y¹ is a linear or branched hydrophilic moiety comprising at least one polymeric moiety independently selected from poly(alkylene oxides) and polyalcohols; and
Z is a hydrophobic moiety; and
administering an effective amount of the formulation in the mammal to enhance the bioavailability or absorption of the ubiquinol or resveratrol in the mammal by a factor of at least two when compared to the administration of the same quantity of a ubiquinol or a resveratrol in the absence of the absorption enhanced bioactive formulation.

16. The method of claim 15, wherein the enhancement of bioavailability or absorption of the ubiquinol or resveratrol, or mixtures thereof, in the mammal is by a factor of at least two to ten.

17. The method of claim 15, wherein the ubiquinol ester/diester or resveratrol ester is selected from the group consisting of the C₂₋₂₀ alkyl ester, the C₂₋₁₀ alkyl ester, the C₂₋₆ alkyl ester or the C₂₋₃ alkyl ester, and mixtures thereof.

18. The method of claim 15, wherein the solubilizing agent is TPGS (polyoxyethanyl-a-tocopheryl succinate) or TPGS-1000 (polyoxyethanyl-a-tocopheryl succinate-1000).
